(19) United States
(12) Patent Application Publication
(10) Pub. No.: US 2021/0299144 A1
(43) Pub. Date: Sep. 30, 2021

(54) USE OF PREGNENOLONE SULFATE TO REDUCE CYST FORMATION IN KIDNEY

(71) Applicants: University of Cincinnati, Cincinnati, OH (US); Children's Hospital Medical Center, Cincinnati, OH (US)

(72) Inventors: Steven J Kleene, Cincinnati, OH (US); Nancy K Kleene, Cincinnati, OH (US); Bradley P Dixon, Centennial, CO (US); Brian J Siroky, Edgewood, KY (US)

(73) Assignees: University of Cincinnati, Cincinnati, OH (US); Children's Hospital Medical Center, Cincinnati, OH (US)

(21) Appl. No.: 17/265,096

(22) PCT Filed: Aug. 2, 2019
(86) PCT No.: PCT/US2019/044834

§ 371 Date: Feb. 1, 2021

(51) Int. Cl. A61K 31/57; A61K 9/00; A61P 13/12

(52) U.S. Cl. CPC A61K 31/57; A61P 13/12; A61K 9/0053; A61K 9/0019

(57) ABSTRACT

A method for the treatment or the prevention of a kidney disorder is disclosed. The method involves administering an effective concentration of pregnenolone sulfate to a subject to increase cytoplasmic Ca²⁺ in kidney cells.

### CROSS REFERENCE TO RELATED APPLICATIONS

The present application is a continuation of PCT Application No. PCT/US2019/044834, filed on Aug. 2, 2019, which claims the benefit of U.S. Provisional Application Ser. No. 62/713,665, filed Aug. 2, 2018, which application is hereby incorporated by reference in its entirety.

### STATEMENT REGARDING FEDERALLY FUNDED RESEARCH OR DEVELOPMENT

This invention was made with government support under DK074038 awarded by National Institutes of Health. The government has certain rights in the invention.

### TECHNICAL FIELD

The present invention relates to a method to reduce cyst formation in kidneys. More specifically, this invention relates to increasing cytoplasmic Ca²⁺ via the primary cilia.

### BACKGROUND OF THE INVENTION

Polycystic kidney disease (PKD) is an inherited disorder in which clusters of cysts develop primarily within your kidneys, causing your kidneys to enlarge and lose function over time. Cysts are noncancerous round sacs containing fluid. The cysts vary in size, and they can grow very large. Having many cysts or large cysts can damage your kidneys. The disease can cause serious complications, including high blood pressure and kidney failure. Two specific forms of this disease are autosomal dominant polycystic kidney disease (ADPKD) and autosomal recessive polycystic kidney disease (ARPKD).

Autosomal dominant polycystic kidney disease is one of the most common, life-threatening genetic diseases. In ADPKD, the fluid-filled cysts develop and enlarge in both kidneys, eventually leading to kidney failure. It is the fourth leading cause of kidney failure and more than 50 percent of people with ADPKD will develop kidney failure by age 50. Once a person has kidney failure, dialysis or a transplant are the only options.

Autosomal recessive polycystic kidney disease (ARPKD) is a rare genetic disorder characterized by the formation of fluid-filled cysts in the kidneys. It is caused by mutations of the PKHD1 gene. Most affected infants have enlarged kidneys during the newborn (neonatal) period and some cases may be fatal at this time. The severity of the disorder and the specific symptoms that occur can vary greatly from one person to another. Some affected children eventually develop end-stage renal disease sometime during the first decade of life. In some patients, symptoms do not develop until adolescence or even adulthood.

Importantly, ADPKD and ARPKD are pathologies associated with cilia dysfunction, also known as ciliopathies. The primary cilium is a solitary “9+0” microtubule-based, hair-like organelle anchored to the mother centriole and projecting from the surface of mammalian cells. Most cells in the body possess a single primary cilium. Signals generated by primary cilia play critical roles in human health. Defects in primary cilia are implicated in kidney disease, cancer, cognitive impairment, and obesity. While the devastating consequences of ciliary pathologies are clear, the mechanisms of signaling by primary cilia are less well understood. However, an important functional focus has been identified. The primary cilium is specialized for Ca²⁺ signaling. Demonstration of this was advanced by the development of methods for recording electrical signals and intraciliary Ca²⁺ changes in the native cilia.

Using these and other methods, several Ca²⁺-conducting ion channels have now been identified in the membranes of various primary cilia. These channels include TRPC1, TRPM3, TRPP2 (also called polycystin-2, PKD2, or PC2), TRPP3 (also called PKD2-L1), TRPV4, and L-type Ca²⁺ channels. Importantly, defects in TRPP2 are known to cause some cases of autosomal dominant polycystic kidney disease. Therefore, a need still exists for a means to increase cytoplasmic Ca²⁺ by activating the ciliary TRPP2-dependent channel.

### SUMMARY OF THE INVENTION

In testing reagents that modulate TRPM3 activity, we unexpectedly found that some strongly influence a known ciliary TRPP2-dependent channel. We further found that knocking out TRPM3 eliminates the ciliary TRPP2-dependent channel. That channel, in other words, requires expression of both TRPM3 and TRPP2. We have identified an activator of this channel, pregnenolone sulfate. Our data demonstrates that pregnenolone sulfate will be beneficial in treating polycystic kidney disease.

The present invention discloses a method for the treatment or the prevention of a kidney disorder, comprising the step of the administering to a subject an effective concentration of pregnenolone sulfate. In one embodiment, the kidney disorder is autosomal dominant polycystic kidney disease (ADPKD). In another embodiment, the kidney disorder is autosomal recessive polycystic kidney disease (ARPKD).

In one embodiment of the present invention, pregnenolone sulfate is administered by injection. In another embodiment, pregnenolone sulfate is administered orally. In one embodiment, the concentration of pregnenolone sulfate administered to a subject is at least about 1 μM. In another embodiment, the concentration of pregnenolone sulfate administered to a subject ranges between about 1 μM and about 230 μM. In yet another embodiment, the concentration of pregnenolone sulfate administered to a subject ranges between about 1 μM and about 75 μM.

The present invention also discloses a pharmaceutical composition comprising pregnenolone sulfate and a pharmaceutically acceptable excipient. In one embodiment, the concentration of pregnenolone sulfate in the composition ranges between about 1 μM and about 230 μM. In another embodiment, the concentration of pregnenolone sulfate in the composition ranges between about 1 μM and about 75 μM.

The present invention also discloses a method for improving the conductance of renal cilia, comprising the step of exposing the renal cilia to the presence of pregnenolone sulfate at a concentration of at least about 1 μM. In one embodiment, the renal cilia are exposed to the presence of pregnenolone sulfate at a concentration from about 1 μM and about 230 μM. In another embodiment, the concentration of pregnenolone sulfate administered to the renal cilia ranges between about 1 μM and about 75 μM.

### BRIEF DESCRIPTION OF THE DRAWINGS

The objects and advantages of the disclosed invention will be further appreciated in light of the following detailed descriptions and drawings in which:

FIG. 1 is two electrical recordings of ciliary channels from the cilia of mIMCD-3 cells. The top recording had no exposure to pregnenolone sulfate, and the bottom recording shows activation by 230 μM extracellular pregnenolone sulfate.

FIG. 2 is two electrical recordings of ciliary channels. The top recording shows activation by 75 μM extracellular pregnenolone sulfate, and the bottom recording shows the loss of activation on removal of the pregnenolone sulfate.

FIG. 3 is a graph showing a single-channel current-voltage relation of ciliary channels measured in standard external solution without (black) or with (red) 230 μM pregnenolone sulfate.

FIG. 4 is two electrical recordings of ciliary channels. The top recording shows the activity of a single ciliary channel observed while recording in the standard solutions. The bottom recording shows a lack of channel activation in the same cilium 5 min after replacing the cytoplasmic solution with one containing 230 μM pregnenolone sulfate.

FIG. 5 is a graph showing the dependence of channel activation on voltage and pregnenolone sulfate concentration. The cytoplasmic solutions contained 0.1 μM cytoplasmic free Ca²⁺. Channel open probability was measured as a function of membrane potential.

FIG. 6 is a graph showing the dependence of channel activation on voltage and pregnenolone sulfate concentration. The cytoplasmic solutions contained 3 μM cytoplasmic free Ca²⁺. Channel open probability was measured as a function of membrane potential.

FIG. 7 is a graph showing the dependence of channel activation on voltage and pregnenolone sulfate concentration. Data using a standard external solution to which 75 μM of pregnenolone sulfate was added are shown as purple circles. Data using a standard external solution to which 230 μM of pregnenolone sulfate was added are shown as orange circles.

FIG. 8 is a series of recordings showing that isosakuranetin reduces the activity of a ciliary channel. The top recording shows the activity of a single ciliary channel in the absence of isosakuranetin. The middle and bottom recordings show reduced channel activity in the same cilium following transfer to solutions containing 1 μM (5 min) or 10 μM (2 min) isosakuranetin, respectively.

FIG. 9 is a representative western blot of membrane proteins labeled for total protein.

FIG. 10 is a representative western blot of membrane proteins labeled for TRPP2.

FIG. 11 is a graph showing normalized TRPP2 labeling of a wild-type (WT) cell line, a TRPP2-knockout cell line, and two TRPM3-knockout cell lines. The graph demonstrates that membrane protein labeling for TRPP2 in two TRPM3-knockout cell lines is not significantly different from that in the wild-type.

FIG. 12 is a series of representative maximum intensity projections through a Z stack of images for each of the wild-type (WT), TRPP2-knockout (TRPP2 KO), and TRPM3-knockout (TRPM3 KO) cell lines.

FIG. 13 is a box-plot graph of the median intensity of TRPP2 immunolabeling/ciliary volume for each cell line and shows that ciliary labeling for TRPP2 in two TRPM3-knockout cell lines is not significantly different from that in the wild type.

FIG. 14 is a graph showing that cytoplasmic Ca²⁺ increases in response to pregnenolone sulfate and that the increase partially depends on TRPP2 expression.

FIG. 15 is a graph showing measurements of pregnenolone sulfate concentration in a standard external solution.

FIG. 16 is a series of representative single-channel recordings.

FIG. 17 is an image of enlarged 3D-rendered views of cilia to illustrate the processing steps used to quantify TRPP2 ciliary labeling.

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided herein.

While the following terms are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently—disclosed subject matter belongs.

Unless otherwise indicated, all numbers expressing quantities of ingredients, properties such as reaction conditions, and so forth used in the specification and claims are to “about.” Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

An “effective concentration,” as used herein, refers to an amount of a substance (e.g., a therapeutic compound and/or composition) that elicits a desired biological response. In some embodiments, an effective concentration of a substance is an amount that is sufficient, when administered to a subject suffering from or susceptible to a disease, disorder, and/or condition, to treat, diagnose, prevent, and/or delay and/or alleviate one or more symptoms of the disease, disorder, and/or condition. As will be appreciated by those of ordinary skill in this art, the effective concentration of a substance may vary depending on such factors as the desired biological endpoint, the substance to be delivered, the target cell or tissue, etc. For example, the effective concentration of a formulation to treat a disease, disorder, and/or condition is the amount that alleviates, ameliorates, relieves, inhibits, prevents, delays onset of; reduces severity of and/or reduces incidence of one or more symptoms or features of the disease, disorder, and/or condition. Furthermore, an effective concentration may be administered via a single dose or via multiple doses within a treatment regimen. In some embodiments, individual doses or compositions are considered to contain an effective concentration when they contain a concentration effective as a dose in the context of a treatment regimen. Those of ordinary skill in the art will appreciate that a dose or concentration may be considered to be effective if it is or has been demonstrated to show statistically significant effectiveness when administered to a population of patients; a particular result need not be achieved in a particular individual patient in order for a concentration to be considered to be effective as described herein.

The terms “treat,” “treatment,” and “treating,” as used herein, refer to a method of alleviating or abrogating a disease, disorder, and/or symptoms thereof in a subject.

As used herein, a “subject” refers to a mammal. Optionally, a subject is a human or non human primate. Optionally, the subject is selected from the group consisting of mouse, rat, rabbit, monkey, pig, and human. In a specific embodiment, the subject is a human.

As used herein, the term “kidney disorder” means any renal disorder, renal disease, or kidney disease where there is any alteration in normal physiology and function of the kidney.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. “A pharmaceutically acceptable excipient” as used in the specification and claims includes both one and more than one such excipient.

Embodiments of the disclosed invention are directed to the use of pregnenolone sulfate as an effective treatment for polycystic kidney disease. Diseases in this class have several biochemical hallmarks, including lowered cytoplasmic Ca²⁺, elevated intracellular cAMP, cellular hyperproliferation, and excess secretion of fluid. Of these, the reduction in cytoplasmic Ca²⁺ is widely believed to be a root cause of the pathology. In cystic cells from human patients and in a rat model, application of a general Ca²⁺ ionophore or Ca²⁺ channel agonists increased cytoplasmic Ca²⁺ and reversed the cystic phenotype. We have discovered that pregnenolone sulfate activates a small ciliary Ca²⁺ influx via the TRPM3/TRPP2-dependent channel.

In autosomal dominant polycystic kidney disease (ADPKD), the great majority of patients lack functional polycystin-1 (PC1). In that case, expression of TRPP2 may be preserved. In autosomal recessive polycystic kidney disease (ARPKD), the primary defect is in fibrocystin/polyductin. In ADPKD or ARPKD patients who retain functional ciliary TRPP2, pregnenolone sulfate in the filtrate should promote a beneficial ciliary Ca²⁺ influx. Since pregnenolone sulfate is a natural steroid metabolite, an effective and non-toxic dose can be determined.

In the present invention, we disclose that a kidney disorder can be treated or prevented by administering to a subject an effective concentration of pregnenolone sulfate. In one embodiment, the concentration of pregnenolone sulfate administered to a subject is at least about 1 μM. In another embodiment, the concentration of pregnenolone sulfate administered to a subject ranges between about 1 μM and about 230 μM. In yet another embodiment, the concentration of pregnenolone sulfate administered to a subject ranges between about 1 μM and about 75 μM.

In one embodiment, the kidney disorder treated or prevented is autosomal dominant polycystic kidney disease (ADPKD). In another embodiment, the kidney disorder is autosomal recessive polycystic kidney disease (ARPKD). In one embodiment, the pregnenolone sulfate may be administered by injection. In another embodiment, pregnenolone sulfate is administered orally.

The present invention also discloses a pharmaceutical composition comprising pregnenolone sulfate and a pharmaceutically acceptable excipient. In one embodiment, the concentration of pregnenolone sulfate in the composition ranges between about 1 μM and about 230 μM. In another embodiment, the concentration of pregnenolone sulfate in the composition ranges between about 1 μM and about 75 μM.

The present invention also discloses a method for improving the conductance of renal cilia, comprising the step of exposing the renal cilia to the presence of pregnenolone sulfate at a concentration of at least about 1 μM. In one embodiment, the renal cilia are exposed to the presence of pregnenolone sulfate at a concentration from about 1 μM and about 230 μM. In another embodiment, the concentration of pregnenolone sulfate administered to the renal cilia ranges between about 1 μM and about 75 μM.

Regarding renal cilia, the primary cilia of renal epithelial cells express a large-conductance cationic channel. This channel is absent if the TRPP2 channel protein is genetically eliminated. We have discovered that, in the renal epithelial cell line mIMCD-3, this same channel also requires expression of a second channel subunit, TRPM3. Furthermore, the channel displays pharmacological properties characteristic of TRPM3 channels and the ionic selectivity of TRPP2 channels.

In renal primary cilia, both TRPM3 and TRPP2 have been detected by immunocytochemistry. In addition, direct evidence of functional ciliary TRPP2-dependent channels has been gained from electrophysiological studies. However, there has been no comparable evidence for ciliary TRPM3 channels. While it is known that TRPM3 contributes to the response to hyperosmolality in a cilium-dependent fashion, we have seen no stereotypical TRPM3 channels or currents in the cilium, even in the absence of TRPP2. Exogenously expressed TRPM3 has a single-channel conductance of 83 pS when conducting Na^{t}, which is similar to the conductance of the ciliary channel. However, expressed TRPM3 channels often show spontaneous activity, whereas the ciliary channels are only active with depolarization or micromolar levels of cytoplasmic Ca²⁺. TRPM3 channels conduct Na⁺ and K⁺ equally well, while the ciliary channel prefers K⁺ (P_{K}/P_{Na}=2.4 to 7.3). The permeability of TRPM3 to Ca²⁺ varies between the two splice variants that have been characterized. TRPM3α2 is highly permeable to Ca²⁺ (P_{Ca}/P_{Na}≈12), while TRPM3α1 is at least 10 times less conductive to Ca²⁺. The ciliary channel conducts Ca²⁺ only weakly (P_{Ca}/P_{Na}=0.06).

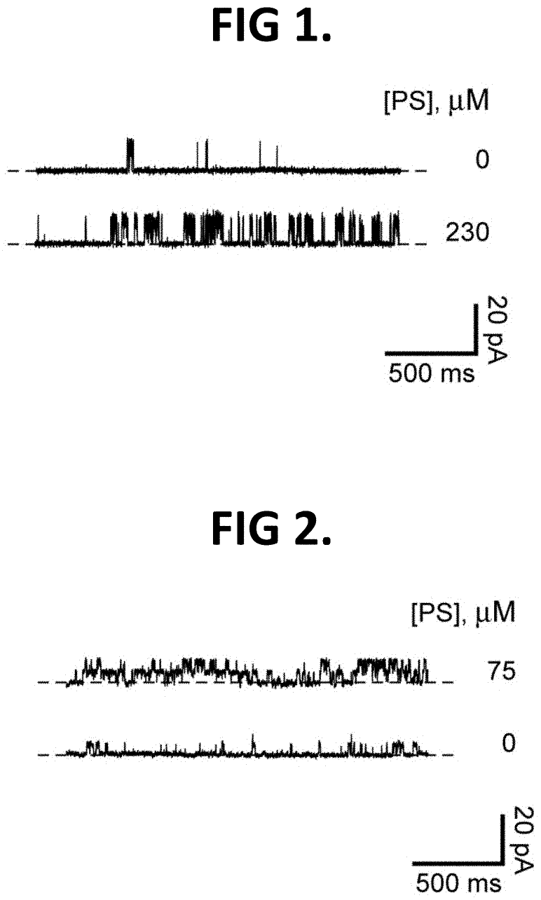

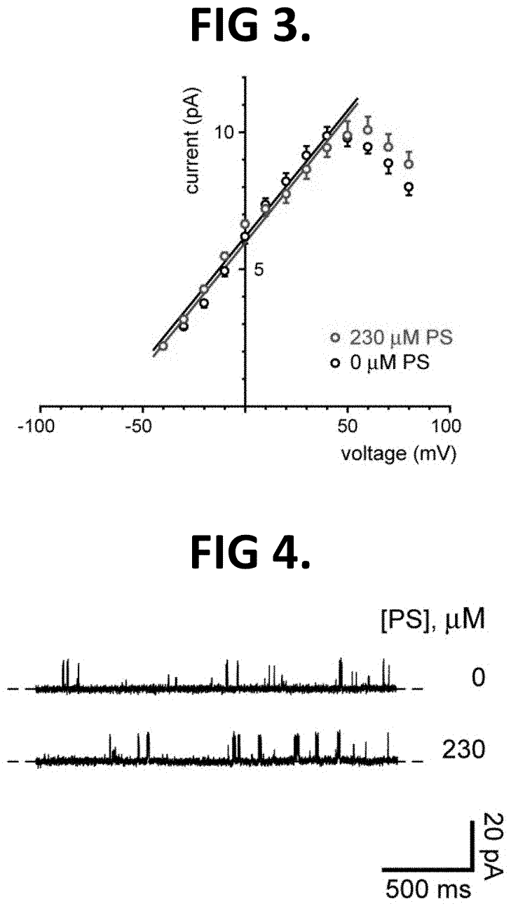

The pharmacological profile of the ciliary TRPP2-dependent channel matches that of TRPM3. The open probability of the ciliary channel is strongly increased by pregnenolone sulfate (FIGS. 1,2,5-7), as occurs with TRPM3. For exogenously expressed TRPM3α2 channels, pregnenolone sulfate has a half-maximal effect at 23 μM (−80 mV) or 12 μM (+80 mV). This closely matches the dependence of the ciliary TRPP2-dependent channel on the concentration of pregnenolone sulfate (FIG. 6). The single-channel current-voltage relation of the TRPP2-dependent channel is not significantly changed by the addition of pregnenolone sulfate (FIG. 3). It is thus unlikely that pregnenolone sulfate is activating a different class of channel. Furthermore, the incidence of large-conductance channels is not increased by pregnenolone sulfate. In the cilium, pregnenolone sulfate is much less effective from the cytoplasmic face of the membrane, as is true for TRPM3 channels (FIG. 4). Finally, the ciliary channel is less active in the presence of isosakuranetin, a selective inhibitor of TRPM3 (FIG. 8).

Without being bound by theory, our hypothesis is that the ciliary channel may be a heteromultimer that includes both TRPM3 and TRPP2 subunits. It is established that TRPP2 can contribute to heteromultimers in renal epithelial cells. TRPP2 colocalizes with TRPC1 in the cilia of renal cells, and coexpression of the two proteins results in a new type of channel. TRPP2 and TRPV4 colocalize in the cilia of another renal cell line and interact functionally in an expression system. A 23-pS channel has been identified in renal cells (although not in the cilia) that depends on both TRPP2 and TRPV4. In mIMCD-3 cells, we have not encountered single channels with the reported properties of TRPC1/TRPP2 or TRPV4/TRPP2 heteromultimers. The large-conductance channel we observe depends on expression of both TRPP2 and TRPM3 and is a pharmacological match to TRPM3.

### Examples

The primary cilia of renal epithelial cells express a large-conductance, cationic channel that is absent unless TRPP2 is expressed. These cilia also express the channel protein TRPM3, but this has only been demonstrated by immunocytochemistry. We examined interdependence between the TRPM3 and TRPP2 channel subunits by recording transmembrane currents in single cilia excised from mIMCD-3 cells.

With reference to FIG. 1, in cilia expressing the large-conductance channel, depolarization to +60 mV caused infrequent openings of the channel (FIG. 1, top recording). When the external (pipette) solution was replaced by perfusing the pipette with a solution to which 230 μM pregnenolone sulfate had been added, the open probability of the channel was greatly increased (FIG. 1, bottom recording). Pregnenolone sulfate is an agonist of TRPM3 channels. On average, external pregnenolone sulfate increased the mean channel current by a factor of 30±12 (n=6). By contrast, perfusions with a control solution lacking pregnenolone sulfate increased the channel current by a factor of 3.3±0.9 (n=7). The activation by pregnenolone sulfate was significant (P=0.028, t-test for independent measures). The activation was reversible. With 75 μM pregnenolone sulfate in the external solution, the channels were very active at −10 mV (FIG. 2, top recording). After replacing the external solution with one lacking pregnenolone sulfate, the fraction of the mean channel current remaining was 0.14±0.08 (FIG. 2, bottom recording; CI 0.22, n=5).

FIG. 1 demonstrates the activation of ciliary channels by pregnenolone sulfate. The top recording shows the activity of a single ciliary channel observed while recording in the standard solutions (with 0.1 μM cytoplasmic free Ca²⁺). The bottom recording shows increased channel activity in the same cilium 5 min after perfusion of the pipette with standard external solution to which 230 μM pregnenolone sulfate had been added. The holding potential was +60 mV for both recordings. The dashed lines indicate the current level when the channel was closed.

Regarding FIG. 2, the top recording shows the activity of two ciliary channels observed with the standard pipette (external) solution to which 75 μM pregnenolone sulfate had been added. The bottom recording shows decreased channel activity in the same cilium 4 min after perfusion of the pipette with the standard external solution, which lacks pregnenolone sulfate. For both recordings, free Ca²⁺ was 3 and the holding potential was −10 mV.

The current-voltage relation of channels activated by 230 μM external pregnenolone sulfate is shown in FIG. 3 (red). The single-channel conductance and extrapolated reversal potential closely match those of the TRPP2-dependent channels measured in the absence of pregnenolone sulfate (FIG. 3, black). The mean single-channel conductance (slope) without pregnenolone sulfate is 94±6 pS, and the extrapolated reversal potential (x-intercept) is −64±4 mV. The values with pregnenolone sulfate are not significantly different (92±3 pS and −67±2 mV; P=0.78 and P=0.54 respectively, t-tests for independent measures). The decrease in conductance at strongly depolarizing potentials (FIG. 3) is characteristic of the TRPP2-dependent channels in the cilia of mIMCD-3 cells.

FIG. 3 shows a single-channel current-voltage relation measured in standard external solution without pregnenolone sulfate (black) or with 230 μM pregnenolone sulfate (red). For all recordings, cytoplasmic free Ca²⁺ was either 0.1 or 3 μM. Each point shown is the mean of measurements in 3 to 5 cilia (without pregnenolone sulfate) or 7 to 10 cilia (with pregnenolone sulfate). Each straight line is fit to the linear portion of its relation (−40 to +50 mV; R² 0.972, P<0.001 without pregnenolone sulfate; R² 0.992, P<0.001 with pregnenolone sulfate).

Application of pregnenolone sulfate did not increase the incidence of the channels (Table 1, wild type). In untreated cilia, the TRPP2-dependent channels were apparent in 34% of cilia tested. In cilia exposed to pregnenolone sulfate, channels were observed in 30% of the cilia (Table 1, wild type). These incidences were not significantly different (P=0.32, Fisher's exact test). The channels were rare in the apical non-ciliary membrane with or without pregnenolone sulfate (Table 1, wild type). The channels are not detectable in the cilia of cells lacking TRPP2. Addition of pregnenolone sulfate did not reveal any channels in the cilia or apical non-ciliary membrane of cells lacking TRPP2 (Table 1, TRPP2 KO).

TABLE 1
Incidences of TRPP2-dependent
channels in apical and ciliary membranes

| | | cell line | | |
|---|---|---|---|---|
| membrane | PS | wild type | TRPP2 KO | TRPM3 KO |
| ciliary | − | 103/304 | 0/36 | 0/45 |
| | + | 77/259 | 0/28 | ND |
| apical | − | 1/22 | ND | ND |
| | + | 3/57 | 0/30 | 0/34 |

In Table 1, the presence or absence of active TRPP2 channels was assessed with the cilium or apical membrane patch exposed to 3 μM cytoplasmic Ca²⁺ and the voltage clamped to +40 mV. If no large-conductance channels were seen to open within 2 min, active TRPP2 channels were judged to be absent. Where indicated, pregnenolone sulfate was included in the external (pipette) solution at either 75 μM or 230 μM. PS, pregnenolone sulfate; KO, knockout; ND, not determined.

Pregnenolone sulfate was much less effective when applied to the cytoplasmic surface of the cilium (FIG. 4). Cytoplasmic pregnenolone sulfate (230 μM) on average increased the mean channel current by a factor of 3.6±0.7 (n=7). This is a significantly smaller increase than seen with 230 μM external pregnenolone sulfate (P=0.030, t-test for independent measures). In both cases cytoplasmic Ca²⁺ was 0.1 μM.

In FIG. 4, the top recording shows the activity of a single ciliary channel observed while recording in the standard solutions. The bottom recording shows channel activity in the same cilium 5 min after replacing the cytoplasmic solution with one containing 230 μM pregnenolone sulfate. Both cytoplasmic solutions contained 0.1 μM free Ca²⁺. The holding potential was +60 mV for both recordings.

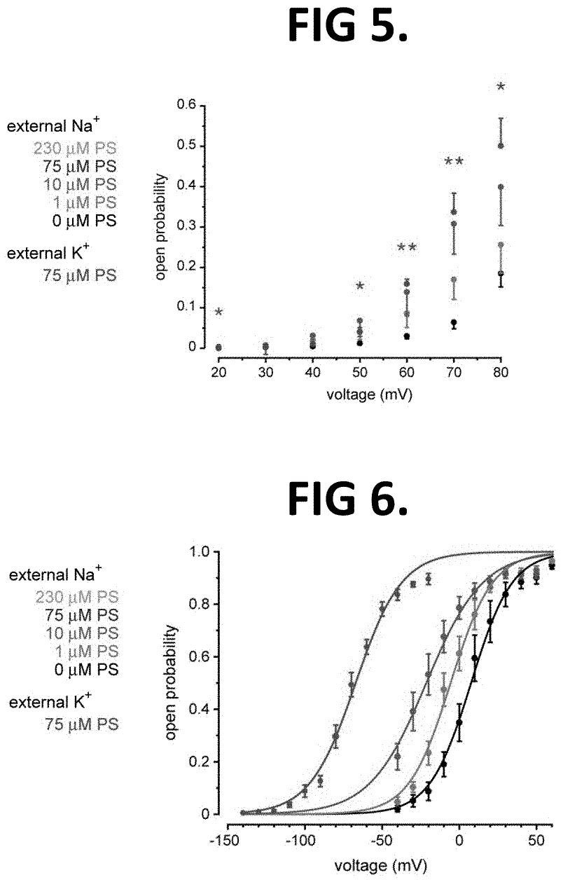

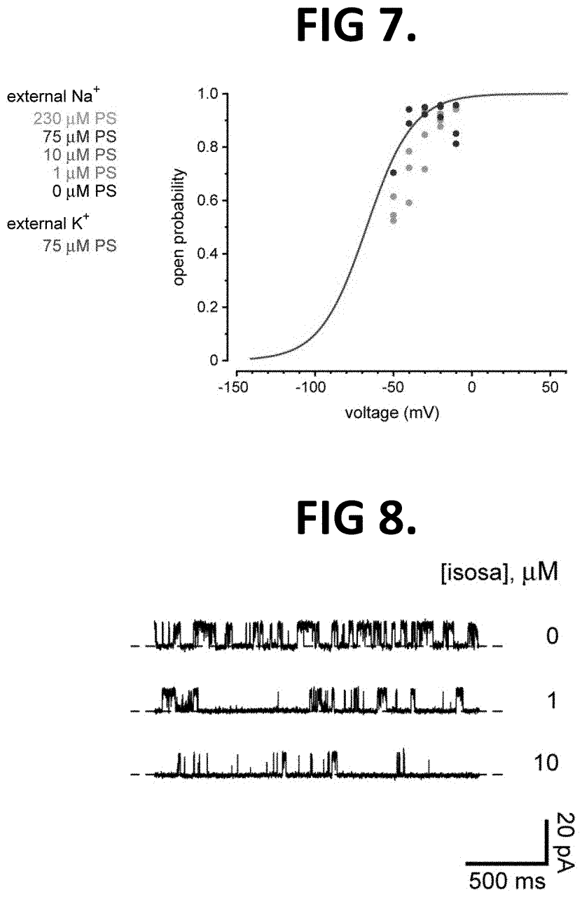

The TRPP2-dependent channel of renal primary cilium is activated by both depolarization and by micromolar levels of cytoplasmic Ca²⁺. When cytoplasmic Ca²⁺ is low (0.1 μM), the channel can be activated by strong depolarization (FIG. 5, black circles). External pregnenolone sulfate significantly increased the channel activity (FIG. 5). Even when the channel was activated by cytoplasmic Ca²⁺, pregnenolone sulfate caused an additional activation. In the presence of 3 μM cytoplasmic Ca²⁺, pregnenolone sulfate at concentrations of 1 to 75 μM significantly shifted the potential for half-maximal activation to more negative values (FIG. 6; one-way ANOVA with Holm-Sidak all pairwise comparison; P<0.001 for all comparisons). Pregnenolone sulfate was no more effective at its solubility limit (230 μM, orange circles) than at 75 μM (red curve, FIG. 7).

To determine the relations shown in FIGS. 5-7, external solutions were chosen that allowed detection of single-channel events at or near the half-maximally effective voltages. For 0 μM, 1 μM and 10 μM pregnenolone sulfate, the standard external solution was used, and the channel current reversed at −67 mV. For 75 μM pregnenolone sulfate, an external solution with KCl instead of NaCl was used, and the channel current reversed near 0 mV. The choice of external solution does not account for the high channel activity in 75 μM pregnenolone sulfate. Even in NaCl-based external solution, channel activity was high at voltages where single-channel events were large enough to be measured (FIG. 7, purple circles).

FIGS. 5-7 demonstrate the dependence of channel activation on voltage and pregnenolone sulfate concentration. In FIG. 5, channel open probability was measured as a function of membrane potential. Cytoplasmic solutions contained 0.1 μM free Ca²⁺. The pipette contained pregnenolone sulfate (0 μM to 75 μM as indicated) dissolved in an external solution that also contained 0.3% (v/v) DMSO. Each point shown is the mean of measurements in 4 to 8 cilia. * Significantly different from 0 pregnenolone sulfate for the given voltage, P<0.001-0.014, t-tests and Mann-Whitney rank sum tests controlled by false discovery rate correction for 21 comparisons. The data in FIG. 6 result from same procedures as in FIG. 5 but with 3 cytoplasmic free Ca²⁺. The external (pipette) solution included pregnenolone sulfate (0 μM to 75 μM as indicated). Each point shown is the mean of measurements in 4 to 11 cilia. The solid smooth curves represent best fits of the open probability-voltage relations to Boltzmann functions. FIG. 7 shows responses in standard external solution to which was added 75 μM (purple circles) or 230 μM (orange circles) pregnenolone sulfate. As in FIG. 6, cytoplasmic solutions contained 3 μM free Ca²⁺. The red Boltzmann function is copied from FIG. 6 (response to 75 μM pregnenolone sulfate with a KCl-based external solution).

TABLE 2
Constants and strength and significance
of fit for the best-fitting Boltzmann functions.

| | Vₘ (mV) | | Boltzmann constants | | | |
|---|---|---|---|---|---|---|
| [PS], μM | tested | n | V_{1/2} (mV) | k (mV) | R² | P |
| 0 | −40 to +60 | 6-9 | 7.6 ± 0.9 | 13.3 ± 0.8 | 0.993 | <0.001 |
| 1 | −40 to +60 | 7-10 | −5.6 ± 1.1 | 13.5 ± 1.0 | 0.990 | <0.001 |
| 10 | −40 to +50 | 9-11 | −21.4 ± 1.4 | 18.3 ± 1.5 | 0.979 | <0.001 |
| 75 | −140 to −20 | 4-9 | −67.2 ± 1.0 | 14.8 ± 0.9 | 0.992 | <0.001 |

For each of four concentrations of external pregnenolone sulfate, the relation between open probability and voltage (FIG. 6) was fit to a Boltzmann function (see Table 2). The concentration of cytoplasmic Ca²⁺ was 3 μM. For each function, the Boltzmann constants V_{1/2} and k are shown, as well as the strength (R²) and significance (P) of the fit. Vₘ, membrane potential; V_{1/2}, the potential at which open probability is 0.5; k, a slope factor; PS, pregnenolone sulfate. Pregnenolone sulfate at concentrations of 1 to 75 μM significantly shifted V_{1/2} to more negative values (one-way ANOVA on V_{1/2} with Holm-Sidak all pairwise comparison; P<0.001 for all comparisons). The data shown in FIG. 5 (0.1 μM free Ca²⁺) were insufficient to define Boltzmann functions.

Pregnenolone sulfate activates the TRPP2-dependent channels but is best known as an agonist of TRPM3. For that reason, we also tested isosakuranetin, a specific inhibitor of TRPM3 that acts from the cytoplasmic face of the membrane. At +30 mV in the presence of 3 μM cytoplasmic Ca²⁺ (and with no pregnenolone sulfate present), the TRPP2-dependent channel opened frequently (FIG. 8, top recording). Addition of cytoplasmic isosakuranetin greatly reduced the open probability of the TRPP2-dependent channel (FIG. 8, middle and bottom recordings). The fraction of the mean channel current remaining was 0.41±0.09 with 1 isosakuranetin (CI 0.23, n=7) and 0.09±0.04 with 10 μM isosakuranetin (CI 0.09, n=8). Inhibition by 10 μM isosakuranetin was not reversible. After 10 min in solution lacking the inhibitor, the fraction of the original uninhibited current never exceeded 0.09 (n=4).

FIG. 8 shows that isosakuranetin reduces the activity of a ciliary channel. The top recording shows the activity of a single ciliary channel in the absence of isosakuranetin. The middle and bottom recordings show reduced channel activity in the same cilium following transfer to solutions containing 1 μM (5 min) or 10 μM (2 min) isosakuranetin, respectively. In all cases cytoplasmic free Ca²⁺ was 3 μM and the holding potential was +30 mV. Isosa, isosakuranetin.

The large-conductance ciliary channel is not detectable unless TRPP2 is expressed. Given the pharmacological similarities between the ciliary TRPP2-dependent channel and TRPM3, we investigated whether the ciliary channel also requires expression of TRPM3. We used an mIMCD-3 cell line in which TRPM3 was knocked out by CRISPR/Cas9 genome editing. In electrical recordings from the primary cilia of cells lacking TRPM3, the large-conductance channel was never seen (n=45, Table 1, TRPM3 KO). This is significantly different from the channel's incidence in cilia from wild-type cells (34%, Table 1, wild type; P<0.001, Fisher's exact test). TRPM3-knockout cells also showed no such channels in the apical non-ciliary membrane even in the presence of pregnenolone sulfate (Table 1, TRPM3 KO). Knocking out TRPM3 had no effect on the activity of another ciliary channel, TRPM4. In 17 cilia tested from TRPM3-knockout cells, 1 mM cytoplasmic Ca²⁺ on average activated a current at +100 mV of 47±7 pA. This is not significantly different from the activity in wild-type cells (50±5 pA, n=88; P=0.65, Mann-Whitney). As in cilia of wild-type cells, 2 mM cytoplasmic MgATP blocked the TRPM4 current. In cilia lacking TRPM3, the fraction of TRPM4 current remaining after addition of MgATP was 0.34±0.03 (CI 0.07, n=16).

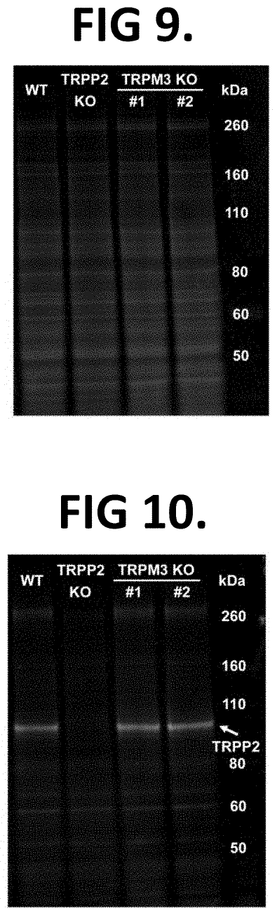

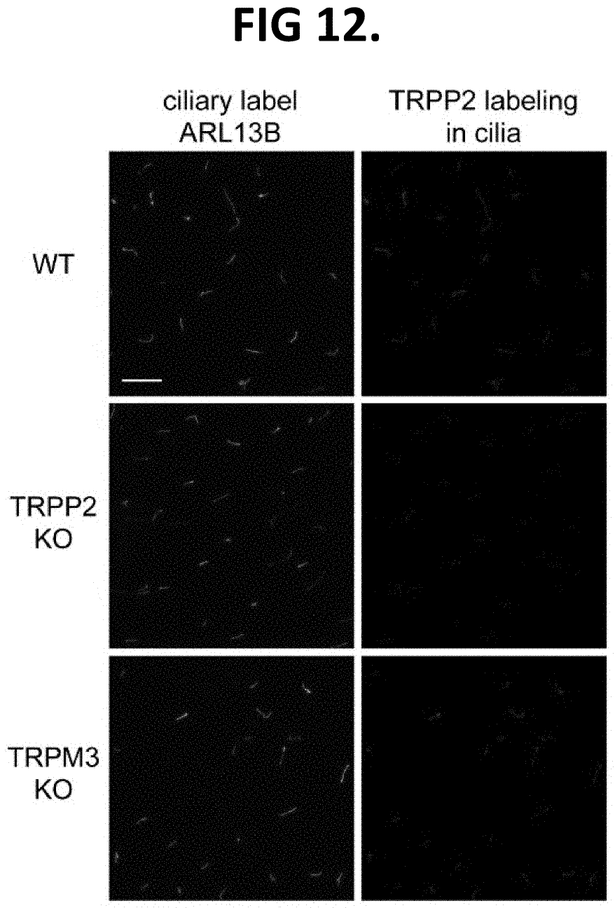

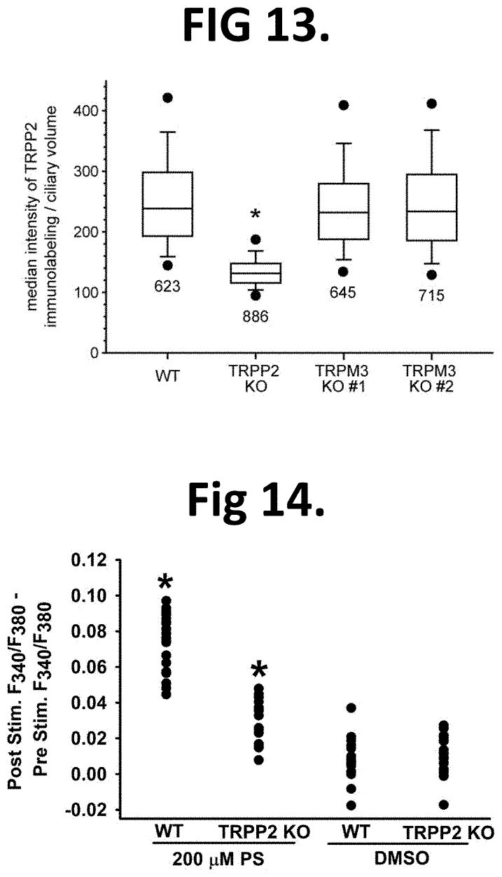

The dependence of functional ciliary TRPP2-dependent channels on TRPM3 expression could suggest that TRPM3 is required for trafficking of TRPP2 to the cilium. Expression of TRPP2 in either of two TRPM3-knockout lines was not significantly changed as judged by western blotting of the membrane protein fraction (FIGS. 9-11). As judged by immunocytochemistry, cells lacking TRPM3 were found to retain expression of TRPP2 protein in the cilia (FIGS. 12 and 13).

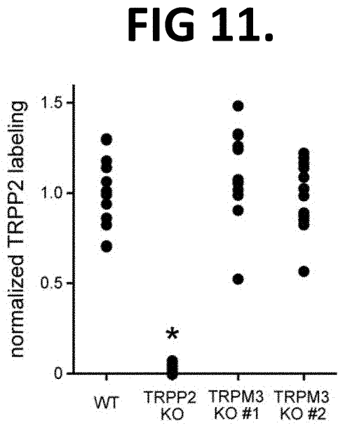

FIGS. 9-11 demonstrate that knockout of TRPM3 does not change TRPP2 membrane protein levels. FIGS. 9 and 10 show representative western blot of membrane proteins labeled for total protein (FIG. 9) and TRPP2 (FIG. 10). TRPP2 is present in wild-type (WT) and TRPM3-knockout (TRPM3 KO) cell lines but absent from the TRPP2-knockout (TRPP2 KO) cell line. Each blot had a duplicate set of lanes also used for analysis but not shown in the figure. FIG. 11 shows quantification of TRPP2 labeling. Membrane protein labeling for TRPP2 in two TRPM3 knockout cell lines is not significantly different from that in the wild type (P=0.75 and P>0.99, one-way ANOVA with Tukey's all pairwise comparison). The TRPP2 knockout has less labeling than the other lines (* P<0.001). Six cell passages of the four cell lines were tested with one blot per passage and two lanes per cell line on each blot. The fluorescent signal of each lane's TRPP2 band was normalized by dividing by a lane normalization factor, which was the Revert total protein stain's fluorescent signal for that entire lane divided by the Revert total protein stain's fluorescent signal for the brightest lane on the blot. To obtain the normalized TRPP2 labeling value (y-axis), each lane's ratio of TRPP2 signal/lane normalization factor was divided by the average of the TRPP2 signal/lane normalization factor for the two lanes containing the wild-type samples for that blot.

FIGS. 12 and 13 show that ciliary TRPP2 immunolabeling is not affected by knocking out TRPM3. In FIG. 12, for each of the wild-type (WT), TRPP2-knockout (TRPP2 KO), and TRPM3-knockout (TRPM3 KO) cell lines, representative maximum intensity projections through a Z stack of images are shown. The cells were immunolabeled for ARL13B, a ciliary protein (first column). The ARL13B labeling was used to define ciliary volumes in the 3D data set as described in Materials and Methods. The maximum intensity projection for the TRPP2 labeling in those ciliary volumes (second column) shows that knocking out TRPM3 does not alter the TRPP2 ciliary labeling. Knocking out TRPP2 reduces the TRPP2 ciliary fluorescence but non-specific labeling remains. Bar=10 μm. All images had contrast enhanced in the same manner. A few brightly labeled pixels were saturated so that lightly labeled pixels would be visible while keeping gamma at 1. No pixels were saturated for the quantification of intensity used to make the graph in FIG. 13. FIG. 13 shows a box plot of the median intensity of TRPP2 immunolabeling/ciliary volume for each cell line. Knocking out TRPM3 does not alter the TRPP2 ciliary labeling (P=0.62 and P>0.99, Kruskal-Wallis one-way ANOVA on ranks with Dunn's all pairwise comparison). Knocking out TRPP2 reduces the TRPP2 ciliary fluorescence by about half compared to the other lines (* P<0.001). The remaining fluorescence in the TRPP2 knockout is considered non-specific labeling by the non-affinity purified, polyclonal TRPP2 antibody, which had several non-specific bands in a western blot of whole-cell lysate from these same wild-type and TRPP2-knockout cell lines as described in Kleene S J, Kleene N K: “The native TRPP2-dependent channel of murine renal primary cilia” Am J Physiol Renal Physiol. 2017; 312: F96-F108. The box plot's top, middle, and bottom lines indicate 75th, 50th, and 25th percentile values, respectively. Whiskers indicate 90% and 10% values; circles indicate the 95% and 5% values. The numbers below each box plot indicate the number of ciliary volumes followed. Four cell passages were used for each cell line.

FIG. 14 shows that pregnenolone sulfate increased cytoplasmic Ca²⁺ in murine renal cells in a manner that is partially dependent on TRPP2. Wild-type (WT) mIMCD-3 cells and a clone of mIMCD-3 cells that lacked TRPP2 (TRPP2 KO) were grown to confluence in a 96-well plate and loaded with Fura2-AM, a ratiometric Ca²⁺ indicator dye. The ratio of the fluorescence (after background subtraction) at 340 nm and 380 nm (F₃₄₀/F₃₈₀) correlates with the cytoplasmic Ca²⁺ concentration. Cells without TRPP2 had a smaller response to 200 μM pregnenolone sulfate (PS, dissolved in 0.4% DMSO) than wild-type cells, and both cell types responded to pregnenolone sulfate more than to 0.4% DMSO (* different from the other three groups P<0.001, ANOVA-Holm-Sidak all-pairwise comparison). Unlike in the electrophysiological ciliary recordings, here the apical surface of the cell, including the cilium, is exposed to the pregnenolone sulfate. Response size (Post Stim. F₃₄₀/F₃₈₀−Pre Stim. F₃₄₀/F₃₈₀) was the average F₃₄₀/F₃₈₀ for 6 time points (14.5-17 min) after the addition of pregnenolone sulfate minus the average F₃₄₀/F₃₈₀ for 6 time points (2.5-0 min) before the addition of pregnenolone sulfate. Responses from individual wells are shown as black circles. The experiment was done with two different passages for each cell line for a total of 19 wells per condition.

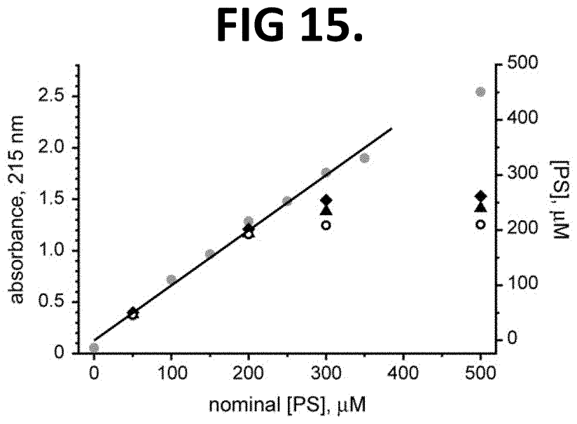

FIG. 15 shows measurements of pregnenolone sulfate concentration in standard external solution. The relation between concentration and absorbance at 215 nm (black line) was established in aqueous samples (gray circles). Concentrations were also measured with pregnenolone sulfate in external solution at nominal concentrations of 50, 200, 300, and 500 μM at times of 10 min (diamonds), 30 min (triangles), or 60 min (open circles). PS, pregnenolone sulfate.

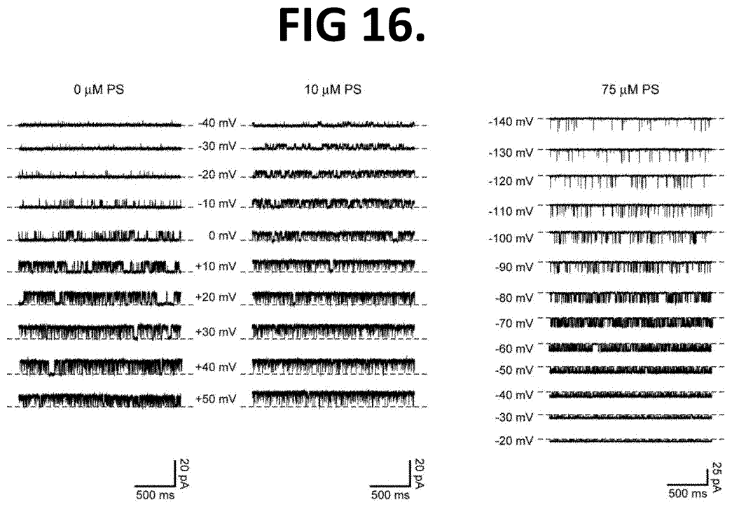

FIG. 16 shows representative single-channel recordings. Recordings show the activity of single ciliary channels observed while recording in the standard KCl-based internal solution plus 3 μM cytoplasmic free Ca²⁺ and the membrane potentials shown. The concentration of external pregnenolone sulfate (PS) is shown above each set (column). Each set was obtained in one cilium. From many such sets, the open probabilities were averaged and shown in FIG. 6. The dashed lines indicate the current level when the channel was closed. External solutions were chosen as described in the text. For 0 μM and 10 μM pregnenolone sulfate, the standard NaCl-based external solution was used, and the channel current reversed at −67 mV. For 75 μM pregnenolone sulfate, a KCl-based external solution was used, and the channel current reversed near 0 mV.

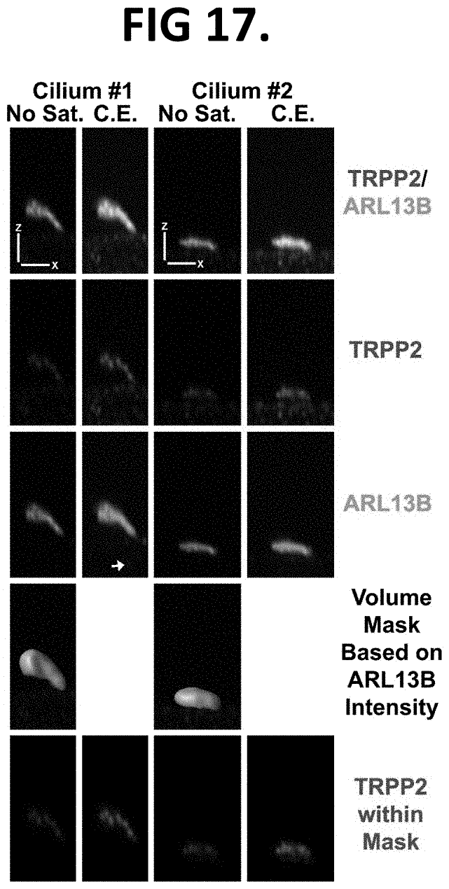

FIG. 17 shows enlarged 3D-rendered views of cilia to illustrate the processing steps used to quantify TRPP2 ciliary labeling. The first row of images shows the overlay of TRPP2 (red) and ARL13B (green) immunolabeling. The second row of images shows just the TRPP2 immunolabeling. The third row shows just the ARL13B immunolabeling. The fourth row shows the volume (gray) that was formed by identifying all the voxels that had an ARL13B intensity value at the defined threshold or greater. The volume image is overlaying the image from the first row. The fifth row of images shows the TRPP2 immunolabeling that was included in the ARL13B-defined volume. We used the median TRPP2 labeling within this volume to represent the TRPP2 labeling for a given cilium. All images in the first and third columns (No Sat.) had contrast enhanced in the same manner with gamma equal to one and no saturated pixels. All the images in the second and fourth columns (C.E.) had the contrast enhanced to allow saturated pixels and, for green, a gamma greater than one (creating a nonlinear relationship between the actual intensity and the displayed intensity) to show both very bright and very dim intensities. No voxels were saturated for the quantification of intensity used to make the graph in FIG. 13. Cilium #1 is an example of cilia with long axes that project well above the cell and are easily isolated from the cytoplasmic TRPP2 labeling. Cilium #4 is an example of cilia with long axes parallel to the surface of the monolayer that are less easily isolated from the cytoplasmic TRPP2 labeling. The same threshold of ARL13B intensity, which was used to define the ciliary volume, was used for all images used in the quantification. This threshold was a compromise value that missed a few very lightly ARL13B-labeled ciliary parts in order to avoid making the volume on more ARL13B-brightly labeled cilia (cilium #2) so large that substantial cytoplasmic TRPP2 labeling would be included. In the image that shows cilium #1, row 3, note the faint green spherical structure in the cytoplasm (arrow); rarely, one of these spheres was bright enough to be thresholded and was then excluded manually. XYZ scale bars=1 μm.

### Electrical Recording

Electrical recordings were made from primary cilia of mIMCD-3 cells. mIMCD-3 cells (murine epithelial cells from the renal inner medullary collecting duct, (CRL-2123, American Type Culture Collection, Manassas, Va., USA) were cultured on beads that were free to move in the recording chamber. Suction was applied to a recording pipette so that a single primary cilium entered the pipette. If a resistance of at least 1 GΩ formed between the membrane and the pipette, the cilium was excised from the cell. This left the cilium inside the recording pipette in the inside-out configuration. The pipette containing the cilium could then be transferred among different solutions that bathed the cytoplasmic face of the membrane. In one set of experiments (FIGS. 1 and 2), the external solution was replaced by perfusing the pipette (FIG. 14).

During recording, the beads coated with cells were stored in a standard external solution containing (in mM) NaCl 140, KCl 5, CaCl₂ 2, MgCl₂ 2, sodium pyruvate 2, HEPES 5, and D-glucose 9.4, adjusted to pH 7.4 with NaOH. The recording pipettes also contained this solution except as noted. In some experiments pregnenolone sulfate was added to the external solution as noted. The standard cytoplasmic solution contained (in mM) KCl 140, NaCl 5, CaCl₂ 0.7, MgCl₂ 2, HEPES 5, BAPTA 2, and D-glucose 5, adjusted to pH 7.4 with KOH. This solution contained 0.1 μM free Ca²⁺. To make a cytoplasmic solution with 3 μM free Ca²⁺, BAPTA was replaced with dibromoBAPTA, and the total CaCl₂ was increased to 1.4 mM. In buffered solutions, concentrations of free Ca²⁺ were estimated by the method of Bers (see Bers D M: “A simple method for the accurate determination of free [Ca] in Ca-EGTA solutions” Am J Physiol Cell Physiol. 1982; 242: C404-C408), which is herein incorporated by reference in its entirety. Pregnenolone sulfate or isosakuranetin was added to the cytoplasmic solution as noted.

All recordings were done under voltage clamp at room temperature (24° C.). Equipment, software, and technical details, including corrections for liquid junction potentials, were conducted as described in the article: Kleene S J, Kleene N K. “The native TRPP2-dependent channel of murine renal primary cilia” Am J Physiol Renal Physiol. 2017; 312: F96-F108, which is herein incorporated by reference in its entirety. During acquisition, currents were low-pass filtered at 2 kHz and digitized at 5 kHz. Total mean channel current was measured as the mean current minus the current attributed to leak channels. The latter was determined from an amplitude histogram. In each cilium reported, the number of large-conductance channels was between 1 and 5. Channel open probabilities were determined from amplitude histograms and are reported only when the number of channels in the membrane was unambiguous. The presence or absence of active TRPP2 channels was assessed with the cilium or patch exposed to 3 μM cytoplasmic Ca²⁺ and the voltage clamped to +40 mV. If no large-conductance channels were seen to open within 2 min, active TRPP2 channels were judged to be absent. These cilia have a second Ca²⁺-activated channel, TRPM4, but it is not activated by the concentrations of cytoplasmic Ca²⁺ used in this study (0.1 or 3 The half-maximal effect of Ca²⁺ on ciliary TRPM4 occurs at 646 μM at +100 mV and 1166 μM at −100 mV.

### Concentration of Pregnenolone Sulfate

For the electrophysiological studies, pregnenolone sulfate was prepared at nominal concentrations up to 300 μM in standard external solution. The 300 μM stock was sonicated but did not appear by visual inspection to be completely dissolved. In order to determine the maximum stable concentration of pregnenolone sulfate in the external solution, aliquots of pregnenolone sulfate in dimethyl sulfoxide (DMSO) were diluted in external solution to final concentrations of 50, 200, 300, and 500 μM in duplicate, sonicated in a sonication bath (Thermo Fisher Scientific, Waltham, Mass., USA) for 1 min, and left at room temperature (25° C.) for 10, 30, or 60 min. The samples were then centrifuged at 10,000×g for 2 min, and for each an aliquot of the supernatant was diluted 20× with doubly distilled water for analysis. Given the simple nature of the samples, HPLC-UV/Vis was used to determine the pregnenolone sulfate concentrations by adapting the method of Sanchez-Guijo et al. (see Sanchez-Guijo A, Oji V, Hartmann M F, Traupe H, Wudy S A “Simultaneous quantification of cholesterol sulfate, androgen sulfates, and progestagen sulfates in human serum by LC-MS/MS.” J Lipid Res. 2015; 56: 1843-1851), which is herein incorporated by reference in its entirety. In short, 5 of each sample was injected into an HPLC system consisting of an Agilent 1100 HPLC equipped with a membrane solvent degasser, a binary pump, a thermostatted auto sampler, thermostatted column compartment, and diode array UV/Vis flow-cell detector with a 10-mm optical path. An Agilent Zorbax Phenyl column (4.6×250 mm, 5 μm particle size) equipped with a C-18 pre-column cartridge was used with the mobile phases A (10 mM ammonium acetate, pH 7, in 85% water and 15% acetonitrile, v/v) and B (70% methanol and 30% acetonitrile). The total flow rate was 1 mL min⁻¹ with a gradient as follows: 0 min, 80% A/20% B; 20 min, 30% A/70% B; 23 min, 1% A/99% B; 25 min, 1% A/99% B; 28 min, 80% A/20% B. An equilibration time of 10 min was used after each injection, and the absorbance at 215 nm (A₂₁₅) was used for quantification based on the peak height.

A set of calibration standards in doubly distilled water was prepared at 50, 100, 150, 200, 250, 300, 350, and 500 μM. This water-based calibration was used to quantify the relation between pregnenolone sulfate concentration and A₂₁₅ (FIG. 15). A good agreement with the water-based calibration was observed for the concentrations of 50 μM and 200 μM in external solution, while 300 μM or above showed little increase in pregnenolone sulfate concentration. For the experiments shown herein, the highest nominal concentration of pregnenolone sulfate was 300 μM. The true concentration in that case averaged 232±9 μM (FIG. 15) and is reported herein as 230 μM.

### Cell Lines

Cell lines lacking TRPM3 were used based on the methods described in the article: Siroky B J, Kleene N K, Kleene S J, Varnell C D, Jr, Comer R G, Liu J, et al. “Primary cilia regulate the osmotic stress response of renal epithelial cells through TRPM3.” (Am J Physiol Renal Physiol. 2017; 312: F791-F805), which is herein incorporated by reference in its entirety. Cell lines lacking TRPP2 were used based on the methods described in the article: Kleene S J, Kleene N K. “The native TRPP2-dependent channel of murine renal primary cilia.” (Am J Physiol Renal Physiol. 2017; 312: F96-F108), which is herein incorporated by reference in its entirety. Since the mIMCD-3 line is nearly triploid, we note that we sequenced the DNA at the mutation site in the TRPM3-knockout and TRPP2-knockout clones and never saw the wild-type sequence; we only detected mutated sequences.

### Western Blotting

Wild-type mIMCD-3 cells, two mIMCD-3 clones with TRPM3 knocked out, and one with TRPP2 knocked out were grown for 7 to 10 days past confluence on plastic, tissue-culture-treated Petri dishes in DMEM/F12 medium (10-092-CV, Thermo Fisher Scientific) with 10% fetal bovine serum (97068-085, VWR, Radnor, Pa.) and 1% penicillin/streptomycin (30-002-C1, Thermo Fisher Scientific). Cells were rinsed with cold phosphate buffered saline (PBS) three times. PBS contained (in mM) KCl 2.7, KH₂PO₄ 1.5, Na₂HPO₄ 8.1, NaCl 140, pH 7.4. The cells were scraped from the dish and transferred to a centrifuge tube with a small amount of PBS. Cells were pelleted at 190×g/4° C. for 5 min. After removing the supernatant, the cells were resuspended in low ionic strength lysis buffer (10 mM Tris-HCl, 0.5 mM MgCl₂) that contained protease inhibitors (Halt Protease inhibitor cocktail, #87785, Thermo Fisher Scientific) at 10 μL per 1 mL of buffer. The cells were incubated on ice for 10 min and then homogenized with a glass homogenizer. The lysates were centrifuged at 2,000×g/4° C. for 5 min. Membrane protein was isolated by centrifugation of the supernatant at 110,000×g/4° C. for 90 min. The resulting pellet was solubilized in membrane buffer (50 mM Tris-HCl, 150 mM NaCl, 1 mM EDTA, 0.05% sodium deoxycholate, 0.1% sodium dodecyl sulfate, 1% Triton X-100) and protease inhibitors with sonication (15 s with a Cell Disruptor Sonicator (setting 4 on model W-220F, Qsonica, Newtown, Conn.)). The membrane protein was centrifuged at 12,000×g/4° C. for 5 min. Protein concentration of the supernatant was determined with a BCA assay (Thermo Fisher Scientific). Equal amounts (6 μg) of reduced, denatured protein were loaded into wells of a NuPAGE Novex 3-8% Tris-acetate gel (Invitrogen/Thermo Fisher Scientific) and subjected to electrophoresis with sodium dodecyl sulfate at 150 V per Invitrogen's protocol. Proteins were transferred to a PVDF membrane (Immobilon-FL, Fisher Thermo Scientific) for 1 h at 30 V. After air drying, the membrane was labeled with Revert total protein stain per instructions (LI-COR BioSciences, Lincoln, Nebr.), air dried, scanned on an Odyssey CLx Infrared Imaging System (LI-COR BioSciences), and destained. The membrane was blocked in 50% PBS/50% Odyssey blocking buffer (LI-COR BioSciences) and incubated with an antibody to TRPP2 (1/750 dilution, sc-25749, Santa Cruz Biotechnology, Dallas, Tex., USA) overnight at 4° C. per LI-COR instructions. The membrane was incubated with goat anti-rabbit IgG conjugated to Alexa Fluor 790 (1/15,000, A11369, Molecular Probes/Thermo Fisher Scientific). The membrane was air dried and scanned. Empiria Studio software (version 1.0.1.53, LI-COR Biosciences) was used for analysis. Six cell passages of the four cell lines were tested with one blot per passage and two lanes per cell line on each blot. The fluorescent signal of each lane's TRPP2 band was normalized by dividing by a lane normalization factor, which was the Revert total protein stain's fluorescent signal for that entire lane divided by the Revert total protein stain's fluorescent signal for the brightest lane on the blot. To obtain the normalized TRPP2 labeling value, each lane's ratio of TRPP2 signal/lane normalization factor was divided by the average of the TRPP2 signal/lane normalization factor for the two lanes containing the wild-type samples for that blot. We confirmed that 6 μg/lane was in the linear part of the μg/lane vs. signal curve for Revert total protein stain and TRPP2.

### Quantification of Ciliary TRPP2 Immunolabeling

Wild-type mIMCD-3 cells, two mIMCD-3 clones with TRPM3 knocked out, and one with TRPP2 knocked out were grown for 4 to 8 days past confluence on #1.5 coverslips in DMEM/F12 medium (10-092-CV, Thermo Fisher Scientific) with 10% fetal bovine serum (97068-085, VWR or 16000-044, Thermo Fisher Scientific) and 1% penicillin/streptomycin (30-002-C1, Thermo Fisher Scientific) They were fixed with paraformaldehyde with a pH shift, treated with 1% sodium dodecyl sulfate for antigen retrieval, and blocked in a donkey serum-containing buffer. They were sequentially immunolabeled with a rabbit polyclonal anti-TRPP2 antibody (1/250, sc-25749, Santa Cruz Biotechnology) overnight and a mouse monoclonal antibody against ARL13B (ADP-ribosylation factor-like protein 13B, a ciliary marker, 1/1000, N295B/66, Antibodies Incorporated, Davis, Calif., USA) for 1 h. Antibodies were diluted in 1% BSA and 0.02% sodium azide in PBS. The cells were incubated with Alexa Fluor-conjugated secondary antibodies (donkey anti-mouse Alexa Fluor plus 488 (A32766) and donkey anti-rabbit Alexa Fluor 594 plus (A32754), Thermo Fisher Scientific). They were mounted using Prolong Diamond mounting medium (Thermo Fisher Scientific) and allowed to cure for a minimum of 5 d. Oversampled (100× oil objective with 1.45 numerical aperture, 0.06 μm×Y resolution, 0.1 μm Z step, 0.5 Airy unit pinhole, 1.1 μs pixel dwell, 2× integration) three-dimensional stacks of images were acquired on a confocal microscope (A1R, Nikon, Melville, N.Y., USA). The image stacks were coded to blind the investigator and then deconvolved (Lucy-Richardson, 20 iterations, NIS-Elements software, Nikon) to reassign out-of-focus data. The coded, deconvolved stacks were analyzed with Imaris software (Bitplane, Zurich, Switzerland). Here, we used the ARL13B immunolabeling to define each ciliary volume and then determined the median intensity of TRPP2 labeling per ciliary volume (FIG. 17). The same intensity was used as a threshold for ARL13B immunolabeling for all images. Cilia that were not fully captured in the stack were excluded from analysis. Sometimes cilia were not detected as a single volume, so the number of volumes is greater than the number of cilia. Volumes of surface area less than 0.02 μm² were excluded. On occasion, spherical structures that were similar in shape and size to background, cytoplasmic ARL13B labeling reached threshold and then were excluded manually. (See FIG. 17, cilium #1, row 3, column 2 for an example of background, cytoplasmic ARL13B labeling.) All volumes with surface areas greater than 0.02 μm² that were near other ciliary volumes were included because cilia sometimes formed multiple volumes. Four separate passages of all four cell lines were analyzed. Six fields per cell line and passage were examined to generate 96 image stacks.

### Measuring Intracellular Calcium

Wild-type mIMCD-3 cells and one mIMCD-3 clone with TRPP2 knocked out were grown to confluence in a 96-well plate. Standard external solution with 1 mM probenecid was used for all solutions. Cells were loaded with 5 Fura2-AM (a ratiometric, Ca²⁺ concentration-indicator dye), 0.01% pluronic acid, and 0.1% DMSO for 30-38 min and rinsed with a standard external solution before the experiment. The ratio of the fluorescence (after background subtraction) at 340 nm and 380 nm (F₃₄₀/F₃₈₀) indicates the cytoplasmic Ca²⁺ concentration and was measured on a BioTek Synergy 4 plate reader. Background fluorescence (wells with cells, 0.01% pluronic acid, and 0.1% DMSO in standard external solution, but no dye) was subtracted from the F₃₄₀ and F₃₈₀ readings. The response was defined as the average F₃₄₀/F₃₈₀ for six time points (14.5-17 min) after the addition of pregnenolone sulfate minus the average F₃₄₀/F₃₈₀ for six time points (2.5-0 min) before the addition of pregnenolone sulfate. The experiment was done with two different cell passages for a total of 19 wells per condition.

### Materials

Pregnenolone sulfate (sodium salt) and BAPTA were purchased from Sigma-Aldrich (St. Louis, Mo., USA); dibromoBAPTA from Molecular Probes/Thermo Fisher Scientific; and isosakuranetin from Extrasynthese (Genay, Rhone, France). Pregnenolone sulfate and isosakuranetin were diluted from stock solutions in DMSO (100 mM for pregnenolone sulfate and 10 mM for isosakuranetin).

### Statistical Analyses

Statistical analyses were performed with the critical significance level α=0.05. Unless otherwise noted, data are presented as mean and standard error (SE) for n independent observations. Parametric tests were used when the Shapiro-Wilk test for normality was passed, and nonparametric tests were used when it failed. Student's t-tests reported are two-tailed. When data were fit using linear (FIG. 3) or nonlinear (FIG. 6) regression analysis, the results are expressed as the estimates of fit parameters±standard error (SE); R² is the regression coefficient (strength of the fit) and P describes the significance of the fit. CI is the 95% confidence interval.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A method for the treatment or the prevention of a kidney disorder, comprising the step of the administering to a subject an effective concentration of pregnenolone sulfate.

2. The method of claim 1 wherein said kidney disorder is autosomal dominant polycystic kidney disease (ADPKD).

3. The method according to claim 1, wherein said kidney disorder is autosomal recessive polycystic kidney disease (ARPKD).

4. The method according to claim 1, wherein the administration is by injection.

5. The method according to claim 1, wherein the administration is oral.

6. The method according to claim 1, wherein the concentration of pregnenolone sulfate administered to said subject is at least about 1 μM.

7. The method according to claim 1, wherein the concentration of pregnenolone sulfate administered to said subject ranges between about 1 μM and about 230 μM.

8. The method of claim 1, wherein the concentration of pregnenolone sulfate administered to said subject ranges between about 1 μM and about 75 μM.

9. A pharmaceutical composition, comprising pregnenolone sulfate and a pharmaceutically acceptable excipient.

10. The composition of claim 9, wherein the concentration of pregnenolone sulfate in the composition ranges between about 1 μM and about 230 μM.

11. The composition of claim 9, wherein the concentration of pregnenolone sulfate in the composition ranges between about 1 μM and about 75 μM.

12. A method for improving the conductance of renal cilia, comprising the step of exposing the renal cilia to the presence of pregnenolone sulfate at a concentration of at least about 1 μM.

13. The method of claim 12 wherein said renal cilia are exposed to the presence of pregnenolone sulfate at a concentration from about 1 μM and about 230 μM.

14. The method of claim 12 wherein the concentration of pregnenolone sulfate administered to said renal cilia ranges between about 1 μM and about 75 μM.
